(19) United States
(12) Patent Application Publication
(10) Pub. No.: US 2009/0018211 A1
(43) Pub. Date: Jan. 15, 2009

(54) Method for inhibiting of COX2 and inflammation with phenolic aldehydes

(75) Inventors: Muraleedharan G. Nair, Okemos, MI (US); Mulabagal Vanisree, East Lansing, MI (US); Ruby L. Alexander-Lindo, Kingston 6 (JM); David L. DeWitt, Okemos, MI (US)

Correspondence Address: Ian C. McLeod;IAN C. MCLEOD, P.C., 2190 Commons Parkway, Okemos, MI, 48864, US

(73) Assignee: Board of Trustees of Michigan State University, Lansing, MI (US)

(21) Appl. No.: 11/825,748

(22) Filed: Jul. 9, 2007

(51) Int. Cl. A61K 31/11; A61P 29/00

(52) U.S. Cl. 514/699

(57) ABSTRACT

A method for inhibiting of COX2 and inflammation by administering a hydroxybenzaldehyde.

### CROSS-REFERENCE TO RELATED APPLICATIONS

None.

### STATEMENT REGARDING FEDERALLY SPONSORED RESEARCH OR DEVELOPMENT

Not applicable.

### BACKGROUND OF THE INVENTION

(1) Field of the Invention

The present invention relates to the use of hydroxybenzaldehydes (mono-, di- and tri substituted) as COX inhibitors, preferably as COX-2 inhibitors and anti-inflammatory agents.

(2) Description of Related Art

There is no known related art. The mono- and di-hydroxybenzaldehydes (herein hydroxybenzaldehydes) have been synthesized by prior art methods. They are also naturally occurring in Antigonon leptopus, a herb used in food and medicinal preparations.

### OBJECTS

It is therefore an object of the present invention to provide compositions which inhibit COX enzymes and which are effective in reducing inflammation. It is also an object of the present invention to provide natural extracts which can be used as nutraceuticals or phytoceuticals. These and other objects will become increasingly apparent from the following description and the drawings.

### SUMMARY OF THE INVENTION

The present invention relates to a method for inhibiting cyclooxygenase or prostaglandin H synthase enzymes comprising: providing at least one compound hydroxybenzaldehyde selected from the group consisting of mono-, di- and trihydroxybenzaldehydes to inhibit the enzymes. The method can be in vitro. Preferably, the method is in vivo. More preferably, the compound is from Antigonon leptopus. Still further, the hydroxybenzaldehyde is 2,3,4-trihydroxybenzaldehyde which selectively inhibits COX2 enzyme without inhibiting COX1.

The present invention also relates to a method for inhibiting inflammation in a mammal in need thereof comprising: administering at least one compound hydroxybenzaldehyde selected from the group consisting of mono-, di- and trihydroxybenzaldehydes to reduce the inflammation. Preferably, said hydroxybenzaldehyde is obtained from Antigonon leptopus.

### BRIEF DESCRIPTION OF DRAWINGS

FIG. 1 is a graph showing COX-1 and COX-2 enzyme inhibition by hydroxybenzaldehydes at 100 ppm. A-Benzaldehyde (not active); B. 2-hydroxybenzaldehyde; C. 3-hydroxybenzaldehyde; D. 4-hydroxybenzaldehyde; E. 2,3-dihydroxybenzaldehyde; F. 2,4-dihydroxybenzaldehyde; G. 2,5-dihydroxybenzaldehyde; H. 3,4-dihydroxybenzaldehyde; I. 3,5-dihydroxybenzaldehyde; J. 2,3,4-trihydroxybenzaldehyde; K. 2,4,5-trihydroxybenzaldehyde (not active); L. 2,4,6-trihydroxybenzaldehyde; M. 3,4,5-trihydroxybenzaldehyde.

FIG. 2 is a graph showing dose response of compound J,2,3,4-trihydroxybenzaldehyde, compound J, on COX-2 enzyme. This compound was not active on COX-1 enzyme even at a 500 ppm concentration.

### DESCRIPTION OF PREFERRED EMBODIMENTS

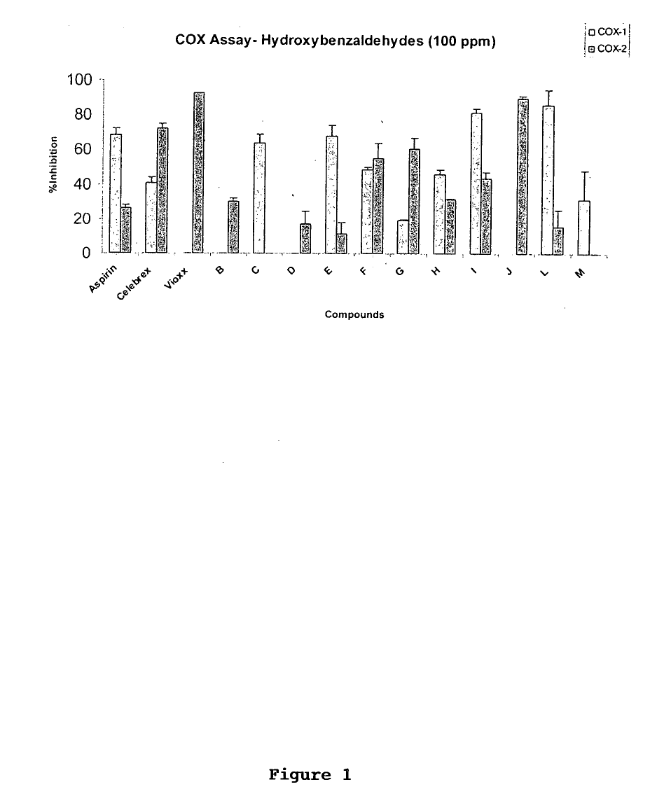

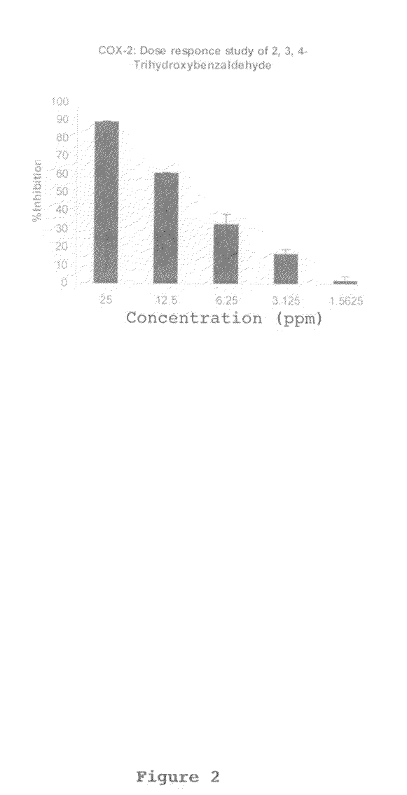

A series of hydroxybenzaldehydes were evaluated for cyclooxygenase enzyme inhibitory activities. The results show that 3-hydroxybenzaldehyde (compound C) selectively inhibited COX-1 enzyme and 2,3,4-trihydroxybenzaldehyde (compound J) selectively inhibited COX-2 enzyme (FIG. 1). The selective inhibition of COX-2 by compound J is potent and an IC₅₀ was observed at around 10 ppm (FIG. 2). Compound J, 2,3,4-trihydroxybenzaldehyde has a great potential to be developed as an anti-inflammatory agent. This compound is present in nature and recently has been isolated from Antigonon leptopus, a herb used in food and medicinal preparations.

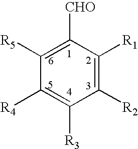

| Compound | R₁ | R₂ | R₃ | R₄ | R₅ |
|---|---|---|---|---|---|
| A | H | H | H | H | H |
| B | OH | H | H | H | H |
| C | H | OH | H | H | H |
| D | H | H | OH | H | H |
| E | OH | OH | H | H | H |
| F | OH | H | OH | H | H |
| G | OH | H | H | OH | H |
| H | H | OH | OH | H | H |
| I | H | H | OH | OH | H |
| J | OH | OH | OH | H | H |
| K | OH | H | OH | OH | H |
| L | OH | H | OH | H | OH |
| M | H | OH | OH | OH | H |

### Cyclooxygenase Inhibitory Assay

Cyclooxygenase enzyme inhibitory assay of the extracts and compounds were carried out using COX-1 and COX-2 enzymes according to the previously published procedures (Jayaprakasam, B. et al., 2006. J. Agric. Food Chem., 54, 5375-5381).

Cyclooxygenase Inhibitory Activity. The rate of oxygen consumption during the initial phase of the enzyme-mediated reaction, with arachidonic acid as substrate was measured using a Model 5300 biological oxygen monitor (Yellow Spring Instrument, Inc., Yellow Spring, Ohio). The test compounds, extracts and positive controls were dissolved in DMSO and an aliquot of 10 μL of each was added to the reaction chamber containing 0.6 mL of Tris buffer (0.1 M, pH 7) and, 1 mM of phenol, hemoglobin (17 μg). COX-1 or -2 enzymes (10 μL) was added to the chamber and incubated for 3 min. The reaction was initiated by the addition of arachidonic acid (10 μL of 1 mg/mL solution). Instantaneous inhibition was measured by using Quick Log Data acquisition and control computer software (Strawberry Tree Inc., Sunnyvale, Calif., USA). The percent inhibition was calculated with respect to DMSO control. Each sample was assayed twice and the standard deviation was calculated for n=2. Aspirin, Celebrex and Vioxx were used as positive controls.

In pharmaceutical compositions, the hydroxybenzaldehyde is inhibitory at a dosage of 1 to 1,000 micrograms per milliliter or gram. In a preferred embodiment, one or more of the hydroxybenzaldehydes for treating a patient are provided to the patient at an inhibitory dose in a pharmaceutically acceptable carrier. As such, the hydroxybenzaldehydes are processed with pharmaceutical carrier substances by methods well known in the art such as by means of conventional mixing, granulating, coating, suspending and encapsulating methods, into the customary preparations for oral or rectal administration. Thus, hydroxybenzaldehyde preparations for oral application can be obtained by combining one or more of the hydroxybenzaldehyde with solid pharmaceutical carriers; optionally granulating the resulting mixture; and processing the mixture or granulate, if desired and/or optionally after the addition of suitable auxiliaries, into the form of tablets or dragee cores.

Suitable pharmaceutical carriers for solid preparations are, in particular, fillers such as sugar, for example, lactose, saccharose, mannitol or sorbitol, cellulose preparations and/or calcium phosphates, for example, tricalcium phosphate or calcium hydrogen phosphate; also binding agents, such as starch paste, with the use, for example, of maize, wheat, rice or potato starch, gelatine, tragacanth, methyl cellulose, hydroxypropylmethyl cellulose, sodium carboxymethyl cellulose and/or polyvinylpyrrolidone, esters of polyacrylates or polymethacrylates with partially free functional groups; and/or, if required, effervescent agents, such as the above-mentioned starches, also carboxymethyl starch, cross-linked polyvinylpyrrolidone, agar, or alginic acid or a salt thereof, such as sodium alginate. Auxiliaries are primarily flow-regulating agents and lubricating agents, for example, silicic acid, talcum, stearic acid or salts thereof, such as magnesium stearate or calcium stearate. Dragee cores are provided with suitable coatings, optionally resistant to gastric juices, whereby there are used, inter alia, concentrated sugar solutions optionally containing gum arabic, talcum, polyvinylpyrrolidone, and/or titanium dioxide, lacquer solutions in aqueous solvents or, for producing coatings resistant to stomach juices, solutions of esters of polyacrylates or polymethacrylates having partially free functional groups, or of suitable cellulose preparations such as acetylcellulose phthalate or hydroxypropyl-methylcellulose phthalate, with or without suitable softeners such as phthalic acid ester or triacetin. Dyestuffs or pigments may be added to the tablets or dragee coatings, for example for identification or marking of the various doses of active ingredient.

Hydroxybenzaldehyde preparation comprising one or more of the hydroxybenzaldehydes which can be administered orally further include hard gelatine capsules, as well as hard or soft closed capsules made from gelatine and, if required, a softener such as glycerin or sorbitol. The hard gelatine capsules can contain one or more of the hydroxybenzaldehydes in the form of a granulate, for example in admixture with fillers such as maize starch, optionally granulated wheat starch, binders or lubricants such as talcum, magnesium stearate or colloidal silicic acid, and optionally stabilizers. In closed capsules, the one or more of the hydroxybenzaldehydes is in the form of a powder or granulate; or it is preferably present in the form of a suspension in suitable solvent, whereby for stabilizing the suspensions there can be added, for example, glycerin monostearate.

Other hydroxybenzaldehyde preparations to be administered orally are, for example, aqueous suspensions prepared in the usual manner, which suspensions contain the one or more of the hydroxybenzaldehydes in the suspended form and at a concentration rendering a single dose sufficient. The aqueous suspensions either contain at most small amounts of stabilizers and/or flavoring substances, for example, sweetening agents such as saccharin-sodium, or as syrups contain a certain amount of sugar and/or sorbitol or similar substances. Also suitable are, for example, concentrates or concentrated suspensions for the preparation of shakes. Such concentrates can also be packed in single-dose amounts.

Suitable hydroxybenzaldehyde preparations for rectal administration are, for example, suppositories consisting of a mixture of one or more of the hydroxybenzaldehydes with a suppository foundation substance. Such substances are, in particular, natural or synthetic triglyceride mixtures. Also suitable are gelatine rectal capsules consisting of a suspension of the one or more of the hydroxybenzaldehydes in a foundation substance. Suitable foundation substances are, for example, liquid triglycerides, of higher or, in particular, medium saturated fatty acids.

Likewise of particular interest are preparations containing the finely ground one or more of the hydroxybenzaldehydes, preferably that having a median of particle size of 5 μm or less, in admixture with a starch, especially with maize starch or wheat starch, also, for example, with potato starch or rice starch. They are produced preferably by means of a brief mixing in a high-speed mixer having a propeller-like, sharp-edged stirring device, for example with a mixing time of between 3 and 10 minutes, and in the case of larger amounts of constituents with cooling if necessary. In this mixing process, the particles of the one or more of the hydroxybenzaldehydes are uniformly deposited, with a continuing reduction of the size of some particles, onto the starch particles. The mixtures mentioned can be processed with the customary, for example, the aforementioned, auxiliaries into the form of solid dosage units; i.e., pressed for example into the form of tablets or dragees or filled into capsules. They can however also be used directly, or after the addition of auxiliaries, for example, pharmaceutically acceptable wetting agents and distributing agents, such as esters of polyoxyethylene sorbitans with higher fatty acids or sodium lauryl sulphate, and/or flavoring substances, as concentrates for the preparation of aqueous suspensions, for example, with about 5- to 20-fold amount of water. Instead of combining the hydroxybenzaldehyde/starch mixture with a surface-active substance or with other auxiliaries, these substances may also be added to the water used to prepare the suspension. The concentrates for producing suspensions, consisting of the one or more of the hydroxybenzaldehyde/starch mixtures and optionally auxiliaries, can be packed in single-dose amounts, if required in an airtight and moisture-proof manner.

In addition, the one or more hydroxybenzaldehydes can be administered to a patient intraperitoneally, intranasally, subcutaneously, or intravenously. In general, for intraperitoneal, intranasal, subcutaneous, or intravenous administration, one or more of the hydroxybenzaldehydes are provided by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the one or more hydroxybenzaldehydes are provided in a composition acceptable for intraperitoneal, subcutaneous, or intravenous use in warm-blooded animals or humans. For example, such compositions can comprise a physiologically acceptable solution such as a buffered phosphate salt solution as a carrier for the one or more hydroxybenzaldehyde. Preferably, the solution is at a physiological pH. In particular embodiments, the composition is injected directly into the patient perfused through the tumor by intravenous administration.

Preparations according to the present invention comprise one or more of the hydroxybenzaldehydes at a concentration suitable for administration to warm-blooded animals or humans which concentration is, depending on the mode of administration, between about 0.3% and 95%, preferably between about 2.5% and 90%. In the case of suspensions, the concentration is usually not higher than 30%, preferably about 2.5%; and conversely in the case of tablets, dragees and capsules with the one or more of the hydroxybenzaldehyde, the concentration is preferably not lower than about 0.3%, in order to ensure an easy ingestion of the required doses of the one or more hydroxybenzaldehydes. The treatment of patients with the preparations comprising one or more of the hydroxybenzaldehydes is carried out preferably by one or more administrations of a dose of the one or more hydroxybenzaldehyde which over time is sufficient to substantially inhibit COX-2. If required, the doses can be administered daily or divided into several partial doses which are administered at intervals of several hours. In particular cases, the preparations can be used in conjunction with or following one or more other therapies such as radiation or chemotherapy. The administered dose of the one or more hydroxybenzaldehydes is dependent both on the patient (species of warm-blooded animal or human) to be treated, the general condition of the patient to be treated, and on the type of disease to be treated.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

1. A method for inhibiting cyclooxygenase or prostaglandin H synthase enzymes comprising:
providing at least one compound hydroxybenzaldehyde selected from the group consisting of mono-, di- and trihydroxybenzaldehydes, but not 4-hydroxybenzaldehyde, to inhibit the enzymes.

2. The method of claim 1 wherein the method is in vitro.

3. The method of claim 1 wherein the method is in vivo.

4. The method of any one of claims 1, 2 or 3 wherein the compound is from Antigonon leptopus.

5. The method of claim 1 wherein the hydroxybenzaldehyde comprises 2,3,4-trihydroxybenzaldehyde and COX2 is selectively inhibited without inhibiting COX1.

6. A method for inhibiting inflammation in a mammal in need thereof comprising:
administering at least one compound hydroxybenzaldehyde selected from the group consisting of mono-, di- and trihydroxybenzaldehydes to reduce the inflammation.

7. The method of claim 6 wherein said hydroxybenzaldehyde is obtained from Antigonon leptopus.

8. The method of claim 6 wherein the at least one compound hydroxybenzaldehyde comprises 2,3,4-trihydroxybenzaldehyde.

9. The method of claim 6 wherein the at least one compound hydroxybenzaldehyde comprises 3-hydroxybenzaldehyde.

10. A method for inhibiting cyclooxygenase or prostaglandin H synthase enzymes comprising:
providing at least one compound hydroxybenzaldehyde selected from the group consisting of 2-hydroxybenzaldehyde, 3-hydroxybenzaldehyde, 2,3-dihydroxybenzaldehyde, 2,4-dihydroxybenzaldehyde, 2,5-dihydroxybenzaldehyde, 3,4-dihydroxybenzaldehyde, 3,5-dihydroxybenzaldehyde, 2,3,4-trihydroxybenzaldehyde, 2,4,6-trihydroxybenzaldehyde, 3,4,5-trihydroxybenzaldehyde, and combinations thereof.

11. The method of claim 10 wherein the at least one compound hydroxybenzaldehyde comprises 2,3,4-trihydroxybenzaldehyde and selectively inhibits COX2 compared to COX1.

12. The method of claim 10 wherein the at least one compound hydroxybenzaldehyde comprises 3-hydroxybenzaldehyde and selectively inhibits COX1 compared to COX2.

13. The method of claim 10 wherein the method is in vitro.

14. The method of claim 10 wherein the method is in vivo.

15. The method of claim 10 wherein the compound is from Antigonon leptopus.
